SEQUENCE LISTING (11 sequences)

SEQ ID NO: 1 - DNA, 60 bp, Artificial Sequence
  Description of Artificial Sequence Synthetic oligonucleotide
gccgaattcgaattccgaatatgcaagcgttacgtaacgttacgcaagcttaagcttgcg

SEQ ID NO: 2 - DNA, 60 bp, Artificial Sequence
  Description of Artificial Sequence Synthetic oligonucleotide
cgcaagcttaagcttgcgtaacgttacgtaacgcttgcatattcggaattcgaattcggc

SEQ ID NO: 3 - DNA, 35 bp, Artificial Sequence
  Description of Artificial Sequence Synthetic oligonucleotide
gcccgaatatgcaagcgttacgtaacgttacgcgc

SEQ ID NO: 4 - DNA, 35 bp, Artificial Sequence
  Description of Artificial Sequence Synthetic oligonucleotide
gcgcgtaacgttacgtaacgcttgcatattcgggc

SEQ ID NO: 5 - DNA, 35 bp, Artificial Sequence
  Description of Artificial Sequence Synthetic oligonucleotide
aattatatttataaatatttatattaattttatat

SEQ ID NO: 6 - DNA, 35 bp, Artificial Sequence
  Description of Artificial Sequence Synthetic oligonucleotide
atataaaattaatataaatatttataaatataatt

SEQ ID NO: 7 - DNA, 32 bp, Artificial Sequence
  Description of Artificial Sequence Synthetic oligonucleotide
ccgggcgcccggcccgggcgcgccgggcgcgc

SEQ ID NO: 8 - DNA, 35 bp, Artificial Sequence
  Description of Artificial Sequence Synthetic oligonucleotide
gcgcgcccggcgcgcccgggccggccggcgcccgg

SEQ ID NO: 9 - DNA, 20 bp, Artificial Sequence
  Description of Artificial Sequence Synthetic primer
tgtgtgcccgtctgttgtgt

SEQ ID NO: 10 - DNA, 20 bp, Artificial Sequence
  Description of Artificial Sequence Synthetic primer
gagtcctgcgtcgagagagc

SEQ ID NO: 11 - DNA, 20 bp, Artificial Sequence
  Description of Artificial Sequence Synthetic probe
cagtggcgcccgaacaggga

(19) United States
(12) Patent Application Publication
(10) Pub. No.: US 2006/0134612 A1
(43) Pub. Date: Jun. 22, 2006

(54) Methods and compositions for retroviral preintegration complex-based assays

(76) Inventors: Frederic Bushman, Rose Valley, PA (US); Julie Mullin, San Diego, CA (US)

Correspondence Address: BIOTECHNOLOGY LAW GROUP;C/O PORTFOLIOIP, PO BOX 52050, MINNEAPOLIS, MN, 55402, US

(21) Appl. No.: 11/055,759

(22) Filed: Feb. 9, 2005

Related U.S. Application Data
(60) Provisional application No. 60/543,701, filed on Feb. 10, 2004.

(51) Int. Cl. C12Q 1/70; C12Q 1/68

(52) U.S. Cl. 435/5; 435/6

(57) ABSTRACT

Disclosed are solution-based methods of screening for molecules that modulate the integration of retroviral cDNA into target DNA molecules. These methods can be used to identify molecules that modulate (e.g., inhibit) the integration of unintegrated retroviral cDNA. Such PIC activity modulators may have therapeutic application, for example, to treat or prevent diseases caused by or associated with retroviral infection.

### RELATED APPLICATIONS

This application claims the benefit of and priority to like-titled and commonly-owned U.S. provisional patent application Ser. No. 60/543,701, filed 10 Feb. 2004, which is hereby incorporated by reference for all purposes.

### GOVERNMENT INTEREST

This invention was made with United States government support under grant number AI-34786, awarded by the National Institutes of Health. As a result, the government may have certain rights in the invention.

### FIELD OF THE INVENTION

The present invention relates to antiretroviral agents and methods and compositions for their identification and characterization.

### BACKGROUND OF THE INVENTION

The following discussion of the background of the invention is merely provided to aid the reader in understanding the invention and is not admitted to describe or constitute prior art to the present invention.

Retroviruses are a diverse family of RNA viruses that synthesize a DNA copy of their RNA genome after infection of the host cell. Viruses of this sort infect virtually all organisms, including mammals, and cause a variety of diseases, including malignancies, autoimmune diseases, immunodeficiencies, and anemias. The retroviral genus is generally divided into three subfamilies: oncovirinae (causing sarcomas and leukemias); lentivirinae (causing slow, progressive disorders such as AIDS); and spumavirinae (the pathology of which is currently unknown). Human retroviruses include human T-cell lymphotrophic viruses 1 and 2 (HTLV-1 and HTLV-2), human immunodeficiency viruses 1 and 2 (HIV-1 and HIV-2), and human spumavirus. Other exemplary animal retroviruses include avian leucosis virus, bovine leukemia virus, feline leukemia virus, mouse mammary tumor virus, Moloney murine leukemia virus, and Mason-Pfizer monkey virus.

Retroviral infections begin with an infecting retrovirus introducing a large nucleoprotein complex into the cytoplasm of the host cell. This complex, which is derived from the core of the infecting virion, contains two copies of the viral RNA genome together with a number of viral proteins, including reverse transcriptase and integrase. Reverse transcription of the viral RNA genome occurs within the complex to make a double-stranded DNA copy of the viral genome. The double-stranded DNA product of the reverse transcription event is known as an “unintegrated viral cDNA.” It differs from the viral RNA genome in that it is longer, containing a direct repeat at each end of the cDNA known as the “long terminal repeats” (“LTRs”). These 5′ and 3′ LTRs promote transcription and polyadenylation of viral messenger RNAs (mRNAs) and appear to be essential for integration of the viral genome into the cellular DNA of the host to form a “provirus”. During the course of transiting between the cytoplasm and nucleus prior to integration, the unintegrated viral cDNA remains associated with both viral and cellular proteins in a nucleoprotein complex termed the “preintegration complex” (“PIC”).

Integration of the viral cDNA into the chromosomal DNA of the host to form a provirus is an essential step in the replication cycle of retroviruses. One constituent of the preintegration complex is the viral integrase protein, the key player in the integration of the viral DNA into the host genome. The other components of the preintegration complex that are transported to the nucleus along with the viral cDNA and integrase, and their possible functions, have not been firmly established. The critical DNA cutting and joining events that integrate the viral cDNA into a chromosome, however, are carried out by the integrase protein itself.

The development drugs that inhibit retroviral reverse transcriptase and protease enzymes have demonstrated that therapeutically effective antiretroviral therapies can be developed. Drugs targeted to integrase or other components of the PIC represent attractive additional therapeutic targets, and recent identification of a class of compounds that inhibits HIV replication in vitro by targeting integrase (Hazuda, et al., Science 287, 646-650, 2000) confirms that such compounds may be identified. The bottlenecks in this identification include the dearth of lead compounds to serve as the starting point for drug development, the inability of integrase-only assays to accurately model the in vivo environment where integrase functions as part of a preintegration complex, and the limited throughput and efficiency of current screening methods for compounds that affect PIC function. Recently, however, PIC-based integration assays have been described. See, e.g., U.S. Pat. No. 6,218,181, and Brooun, et al., J. Biol. Chem. 276: 46946-52, 2001. These assays also have shortcomings, however, as one relies on target DNAs reversibly immobilized on a solid support, whereas the other employs a long, concatameric integration substrate.

Clearly there is a need for improved assays for the identification and characterization of compounds that mediate antiretroviral effects by modulating real-world integrase activity. Among the objects of this invention is to provide such assays, as well as compositions for their performance.

### DEFINITIONS

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

A “chaotropic agent” refers to an agent that disrupts hydrogen bonds. This disruption occurs both in proteins and water. Chaotropic agents can thus disrupt intermolecular interactions. Examples of chaotropic agents include guanidine hydrochloride and urea.

As used herein, the term “contacting” refers to any method of exposing a PIC to a free-floating target DNA and/or a test compound in a way that mimics an in vivo interaction under physiological conditions. Unless otherwise indicated, “contacting” refers to those conditions that, in the absence of an inhibitor of integration, allow a unintegrated viral cDNA to be integrated into a target DNA by the PIC being assayed. For example, the PIC-based assays of the invention are preferably performed in an vitro format, wherein the PIC is typically isolated from cells that have been infected with retroviruses (preferably replication defective retroviruses) whose genomes encode a provirus of interest and the PIC is exposed to a test compound under conditions that approximate or are analogous to those that occur physiologically in the nuclei of cells infected with the native form of the retrovirus from which at least the integrase protein of the PIC is derived.

An “engineered” nucleic acid (e.g., a unintegrated viral cDNA or a target DNA) refers to a nucleic acid molecule that has been directly or indirectly manipulated by man to differ from a naturally occurring nucleic acid in one or more ways. For example, in the context of unintegrated viral cDNA, an engineered viral cDNA includes a viral cDNA that lacks one or more coding or regulatory regions essential to generate infectious viral particles in cells containing a provirus, i.e., an integrated copy of the viral cDNA (typically as a result of infection by a replication defective retrovirus comprising an RNA genome encoding the viral cDNA). Here, “lacking” means that the particular coding or regulatory region is non-functional, due, for example, to partial or complete deletion, rearrangement, or other disruption.

A “free-floating” target DNA refers to a target DNA that is not bound or otherwise attached to a surface of the vessel (e.g., the bottom surface of a well in a microtiter plate) in which a reaction involving the target DNA (e.g., an integration assay) occurs. Instead, the target DNA is in solution as part of the liquid phase of the reaction. Unless otherwise indicated, this is not meant, however, to preclude embodiments where one or more target DNA molecules are attached to other molecules, such as multivalent dendrimers, particles, microcarriers, or the like that remain suspended in solution during the course of a reaction.

A “heterologous marker” refers to a molecule the presence of which indicates the occurrence of a particular event. For example, a heterologous marker can be a protein (e.g., green fluorescent protein, luciferase, etc.) encoded by an engineered viral cDNA and the expression of which can be detected using an appropriate assay.

The term “label moiety” refers to a molecule, or group of molecules, used for detection of another molecule. Typically, a label moiety is attached directly to the molecule to be detected or, alternatively, to a probe or other molecule that specifically reacts with the target molecule. Representative examples of label moieties include fluorescent moieties, electrochemical labels, and radioisotopes.

The terms “viral cDNA” and “unintegrated viral cDNA” refer to a double-stranded DNA molecule that can integrated into a DNA substrate or target from an active PIC. When PICs are prepared in cells, the viral cDNA is the product of the reverse transcription of the RNA genome introduced into the infected cell by an infectious retroviral particle. The first strand of such reverse transcription is referred to a complementary DNA, or “cDNA”, of the RNA genome. As described above, in the process of forming the double-stranded DNA, additional sequences such as 5′ and 3′ LTRs are also incorporated. Preferably, a viral cDNA is obtained packaged into a PIC by retrovirus-infected cells. Such a viral cDNA may contain viral sequences only (preferably containing fewer than all of the viral gene sequences needed to produce an infectious retroviral particle in a suitable host cell), or it may be a recombinant molecule that contains both viral and non-viral sequences (e.g., one or marker genes the expression of which is preferably under the control of a non-retroviral promoter).

As used herein, the term “purified” does not require absolute purity. Instead, it represents an indication that the particular molecule (or complex of molecules) interest is in a discrete environment in which abundance (typically measured on a mass basis) relative to other molecules in the composition has been increased by the purification step. By “discrete environment” is meant a single medium, such as a single solution, a single gel, a single precipitate, etc. Purified molecules (e.g., target DNAs) and molecular complexes (e.g., PICs) may be obtained by a number of methods including, for example, laboratory synthesis, chromatography, preparative electrophoresis, centrifugation, precipitation, affinity purification, etc. One or more “purified” polypeptides, polypeptide complexes (e.g., a PIC), and/or nucleic acids of interest are preferably at least 10% of the polypeptide, polypeptide complex, and/or nucleic acid content of the discrete environment. One or more “substantially purified” polypeptides, polypeptide complexes, and/or nucleic acid are at least 50% of the polypeptide, polypeptide complex, and/or nucleic acid content of the discrete environment, more preferably at least 75%, and most preferably at least 95%.

The term “reaction conditions” refers to the conditions under which the PIC assays of the invention are conducted, and under which a target DNA and viral cDNA are contacted. These conditions are those that allow a viral cDNA to be integrated into a target DNA by the particular PIC in the absence of an inhibitory substance. Preferably, such conditions are physiological conditions, where “physiological conditions” refers to conditions that approximate or are analogous to those that occur in nature with respect to the particular chemical reaction(s) under consideration in terms of temperature, ionic strength, pH, the presence of necessary co-factors, etc. In the context of this invention, preferred reaction conditions are the physiological conditions that exist in the nuclei of cells infected naturally by the retroviral species that corresponds to the integrase protein present in the PICs used in the assay.

A “replication defective” retrovirus refers to a virus that, upon infection of a host cell, can not be replicated to produce additional infectious retroviral particles. Retroviruses can be engineered to be replication defective by, for example, engineering the deletion of one or more coding regions from the viral genome. While such engineered genomes can still be packaged to form infectious virions, subsequent infection of suitable host cells does not result in the production of more infectious particles. A viral cDNA that does not encode all of the elements required for the production of infectious retroviruses lacks replicative capacity, in that it can not replicate through the production of infectious retroviruses.

The term “retroviral preintegration complex” refers to a nucleoprotein complex comprising double-stranded retroviral cDNA and protein, which complex typically forms during retroviral infection of cells, although the invention contemplates PICs generated from any source, including de novo in vitro assembly of the various PIC constituents. An “active” preintegration complex refers to a complex that can mediate the integration of a suitable viral cDNA (preferably an engineered viral cDNA) into an integration substrate (e.g., a free-floating target DNA) under appropriate reaction conditions. Examples of such complexes are described elsewhere herein. Other examples may be prepared by those skilled in the art in view of this specification. As is also well known to the artisan, PICs may be fractionated (e.g., by salt extraction; see, e.g., Chen, et al., Proc. Natl. Acad. Sci. USA 95: 15270-74, 1998), resulting in certain PICs that are “inactive,” i.e., they are unable mediate integration of a viral cDNA into a target DNA under the conditions in which an active PIC could modulate integration. Integration-mediating activity, however, can be restored, for example, by mixing the inactive PICs with those component(s) of an active PIC that is(are) missing. Such components can be provided, for example, by a cell extract prepared from a cell line useful in preparing active PICs of the same type. The term “retroviral preintegration complex” is not intended to indicate an individual molecule of PIC, but rather to refer to a population of PICs used in a particular assay. This population may be obtained from a single retrovirus strain, or may be a pool obtained from a plurality of retroviral types. Similarly, active PICs may be prepared (as assembled) to incorporate viral and/or cellular components from species other than those of naturally occurring PICs. In various preferred embodiments, the retroviral PIC used may be obtained from one or more lentivirus strains, oncovirus strains, and/or spumavirus strains. In particularly preferred embodiments, the retroviral PIC is obtained from one or more lentiviruses, more preferably one or more human lentivirus strains, and most preferably one or more HIV strains. Similarly, retroviral PICs are preferably prepared using cell lines (or portions thereof, e.g., the cytoplasm) derived from cells of the species that the particular retrovirus infects in nature. For instance, when the retrovirus to be modeled is a human lentivirus strain (e.g., HIV-1), it is preferred that the PIC be prepared using a human cell line.

The term “target DNA” as used herein refers to a nucleic acid integration substrate into which a viral cDNA can be integrated in accordance with the instant methods. Such substrates are double-stranded DNA molecules, preferably comprised of naturally occurring nucleotides assembled to have a phosphodiester backbone between at least some, and preferably all, of the contiguous bases of the same strand. As described elsewhere herein, a target DNA can be of any size, although in many embodiments a target DNA comprises no more than several thousand (e.g., about 2,000, 3,000, 4,000, and 5,000) nucleotide base pairs, generally fewer than about 2,000 contiguous nucleotide base pairs, preferably fewer than about 500 base pairs, and even more preferably fewer than about 100 contiguous nucleotide base pairs. Particularly preferred are target DNAs comprising fewer than about 100 base pairs, even more preferably less than or equal to about 50 base pairs, and still more preferably less than or equal to about 20 base pairs. Particularly preferred lengths of the strands comprising a duplex target DNA are between about 15 and about 100 contiguous nucleotides. In the context of target DNAs, the term “about” refers to ±10% of a given length. By “contiguous nucleotides” is meant a number of nucleotides joined in a single covalently linked structure. Thus, a duplex DNA comprising 400 nucleotides and having 200 in each strand of the duplex is considered 200 contiguous nucleotide base pairs in length (assuming neither end of the duplex has one or more 5′ or 3′ “overhanging”, or unpaired, nucleotides). By “nucleotides” is meant the traditional “Watson-Crick” nucleotides A, T, G, and C, as well as the numerous nucleotide and nucleoside analogues known in the art, and nucleotides, nucleosides, and analogues conjugated to tag moieties. A target DNA may be single- or double-stranded. If double-stranded, the two strands may be complementary across the entire length of one or both strands, or may include non-complementary or other unpaired regions in one or both strands.

The term “tag moiety” as used herein with regard to DNA molecules refers to any moiety that may be attached to a DNA molecule and used to identify or purify the DNA molecule. One or more tag moieties can be attached at the ends of DNA molecules (5′ and/or 3′) as well as on non-terminal nucleotides.

The term “test molecule” refers to any molecule that can be tested in an integration-based assay according to the invention. Representative classes of test molecules include polypeptides, nucleic acids, aptamers, small molecules, and carbohydrates. The term “polypeptide” refers to a molecule comprised of a polymer of amino acids linked by peptide bonds. This term includes proteins, fusion proteins, oligopeptides cyclic peptides, polypeptide derivatives, antibodies, and antibody fragments. Proteins can be isolated from a natural source, by solid-state peptide synthesis, or by recombinant methods. In this context, “nucleic acid” refers to polymers of polydeoxyribonucleotides (containing 2′-deoxy-D-ribose or modified forms thereof), to polyribonucleotides (containing D-ribose or modified forms thereof), and to any other type of polynucleotide that is an N-glycoside of a purine or pyrimidine bases, or modified purine or pyrimidine bases. The term “aptamer” refers to a single-stranded or double-stranded oligodeoxyribonucleotide, oligoribonucleotide, or modified derivatives that specifically bind to and alter the biological function of a target molecule. In such contexts, the target molecule is a protein, lipid, carbohydrate, nucleic acid, or a derivative of any of the foregoing. An aptamer is capable of binding a target molecule under physiological conditions. The term “polysaccharide” as used herein refers to a molecule comprising at least 10 glycosidically linked monosaccharide residues, while the term “oligosaccharide” refers to a molecule comprising from 2-10 glycosidically linked monosaccharide residues. The term “small molecule” includes any molecule having a molecular weight less than about 5,000 daltons (Da), preferably less than about 2,500 Da, more preferably less than 1,000 Da, and most preferably less than about 500 Da.

### SUMMARY OF THE INVENTION

The present invention relates in part to compositions and methods for PIC-based integration assays. The compositions and methods described in detail hereinafter concern solution-based assays and employ short nucleic acids as integration substrates. As a result, the assays of the instant invention provide for more efficient use of expensive (both in terms of cost and the time required for preparation) reagents, and are readily adaptable to high-throughput screening of pluralities compounds, for example, large, diverse combinatorial libraries. The present invention can also provide for the efficient identification of PIC components that are required for functional integration of retrovirus viral cDNA, thus providing an aid to the identification of novel drug targets within the PIC. Such information can, for example, be used to generate additional PIC assembly assays to identify compounds that modulate (e.g., inhibit) PIC assembly and/or activity. As will be appreciated, these methods and compositions can permit efficient screening of test molecules for the identification and/or characterization of antiretroviral drugs, including lead compounds as well as derivatives of lead compounds that have improved characteristics.

In a first aspect then, the present invention relates to methods of qualitatively or quantitatively detecting or measuring integration of engineered retroviral cDNA into a free-floating target DNA. These methods occur in aqueous reaction environments, and comprise contacting a target DNA in solution with a retroviral PIC under conditions that, in the absence of an integration inhibitor, allow integration of engineered viral cDNA present in an active PIC into the target DNA.

In this aspect, the target DNA, which is designed to permit integration of viral cDNA, usually is linear and can be of any length. For convenience, it is preferred that the target DNA be no longer than necessary, and thus in many embodiments the target DNA is less than about 5,000, 4,000, 3,000, or 2,000 contiguous nucleotide base pairs in length. Preferably, the target DNA also does not contain a repeating nucleotide sequence, and is not immobilized on a substrate, at least not during the contacting step. Rather, the target DNA is in solution when contacted with the PIC of interest. The target DNA may optionally include one or more tag and/or label moieties. Following this contacting step, a detectable signal may be generated that is related to the presence or amount of viral cDNA that has integrated into the target DNAs present in the reaction.

The PICs used in the assays of the present invention comprise an engineered viral cDNA and one or more protein components required PIC-mediated integration activity. PICs that possess such activity are termed “active” PICs, whereas those that lack one more of the protein components required for integration activity are termed “inactive” PICs. Regardless of whether a PIC is active or inactive, the DNA component is engineered such that its subsequent integration into the genome of a suitable host cell does not result the production of infectious retroviral particles. As such, the unintegrated viral cDNA lacks replicative capacity.

As described in detail hereinafter, retroviral PICs that lack replicative capacity may be obtained from any suitable source, including a variety of host cell types, including acutely infected cell lines and chronically infected cell lines, wherein the cells are infected with replication defective virions. Methods for cultivating such cell lines and purification of PICs are well known to those of skill in the art. See, e.g., Rohdewohld, et al., J. Virol. 61: 336-43, 1987; Famet, et al., Proc. Natl. Acad. Sci USA 93: 9742-47, 1996; Bushman, et al., J. Virol. 71: 458-64, 1997; Chen and Engelman, Proc. Natl. Acad. Sci. USA 95: 15270-74, 1998; Li, et al., J. Virol. 72: 2125-31, 1998; U.S. Pat. No. 6,218,181; Brooun, et al., J. Biol. Chem. 276: 46946-52, 2001; and Jin, et al., J. Virol. 76: 5540-47, 2002. Preferably, the cell line used to produce a PIC is from the same species as the cell line that is infected in nature by the retrovirus that corresponds to the integrase protein incorporated into the particular PIC. Exemplary methods are described hereinafter. As will be appreciated, active PICs may also be generated from PICs that previously were inactive (for example, from salt-stripped PICs) or by in vitro assembly, up to and including de novo PIC assembly of isolated, and preferably purified, PIC components. Similarly, techniques for making replication defective retroviruses are known in the art, and can be readily adapted to generate virions that, following infection, yield PICs according to the invention.

Detection of successful integration of viral cDNA into a target DNA may be performed by any suitable method, including nucleic acid amplification methods, Southern blotting, direct detection of labeled viral cDNA, expression of a marker gene from an integrated provirus, etc. Nucleic acid amplification methods are preferred, as primers may be selected to use only successfully integrated DNA as a template, alone or in conjunction with sequences from the target DNA. While the exemplary methods described hereinafter relate to amplification using the polymerase chain reaction (“PCR”), any of the numerous other nucleic acid amplification methods known in the art (e.g., isothermal methods, rolling circle methods, etc.) can be readily adapted for use in the practice of the invention. The skilled artisan will understand that these other methods may be used either in place of, or together with, PCR methods. Prior to detection, unintegrated viral cDNAs are preferably removed. Such removal can be performed by any suitable method. In preferred embodiments, retroviral cDNAs integrated into target DNAs are isolated from unintegrated viral cDNAs through the use of a tag moiety in the target DNA.

The ability to detect integration of viral cDNA into a target DNA allows, for example, the identification and/or characterization of molecules that modulate aspects of the retroviral infection process, particularly the integration of viral cDNA into target DNA. Thus, another aspect of the invention concerns screening methods to identify modulators of retroviral infection, particularly at the level of integration. In these methods, an active retroviral PIC is contacted with one or more test molecules to be screened for the ability to modulate PIC activity prior to or concurrently with contacting the target DNA with the PIC under conditions that allow integration of viral cDNA present in an active PIC into the target DNA. A change in the ability of the PIC to mediate integration of viral cDNA into the target DNA indicates that the test molecule(s) modulate PIC function. In certain embodiments, the change in PIC-mediated integration is determined by comparing the signal obtained to a baseline signal obtained by performing the same method in the absence of the test molecule(s). As will be appreciated, such methods can be used as a primary screen to identify molecules that modulate PIC-mediated integration activity. They can also be used in later screening rounds. Such methods may also be used in conjunction with integration assays that employ other approaches, for example, the use of a purified retroviral integrase protein to mediate integration of a viral cDNA. As an example, a first screening assay may use a purified retroviral integrase protein to mediate integration of viral cDNA. Molecules identified as integration modulators using such methods can then be confirmed as modulators using a PIC-based integration assay according to the invention.

The present invention may also be advantageously used to identify PIC components, as well as compounds that modulate assembly of active PICs. For example, active PICs can be dissociated (e.g., by salt washing) to produce inactive PICs, wherein at least one component of an active PIC is no longer present in the molecular complex. Then, one or more components may be added back to the inactive PICs to determine those components that are critical to PIC function. Such added components can be characterized in order to identify the critical components, i.e., those components whose presence is essential to produce an active PIC. Thus, in another aspect of the invention, the retroviral preintegration complex used in the methods described above is an inactive retroviral preintegration complex. As will be appreciated, such methods further comprise contacting an inactive PIC with one or more test molecules prior to or concurrently with contacting the target DNA with the PIC under conditions that allow integration of viral cDNA present in an active PIC into the target DNA. Integration of the viral cDNA into the target DNA under these conditions indicates reconstitution of an active retroviral PIC.

PIC components identified in this way can also be advantageously in a number of methods. For example, individual components known to reconstitute active PIC function may be contacted with one or more test molecules to be screened for the ability to modulate PIC activity, in an effort to identify modulator substances. Alternatively, individual components known to reconstitute active PIC function may be contacted with one or more molecules known to modulate PIC activity, in an effort to identify the target of the known modulator. This list of applications is not meant to be limiting. In a related aspect, assays that employ assembly or reconstitution of active PICs from PIC components can be used to screen for test molecules that modulate the assembly of active PICs. In such methods, PIC components are contacted with a test molecule species prior to or concurrently with the combining the various PIC components. Test molecules that modulate the ability of PICs to integrate a viral cDNA into a target DNA are identified as modulators of PIC assembly, and hence PIC activity.

The test molecules to be screened in the various assays of the invention as being potential molecules may be selected, for example, from the group consisting of proteins, polypeptides, nucleic acids, small molecules, aptamers, carbohydrates, peptidomimetics, etc. In certain embodiments, such test molecules are selected from one or more combinatorial libraries. Pools of different species of test molecules can be tested in a single assay; alternatively, a single assay reaction may involve the testing of only a single test molecule species. Modulators of PIC function may be inhibitory or stimulatory, i e., they either inhibit or stimulate PIC activity, as measured by an integration assay according to the invention. Once identified, modulators of PIC-mediated integration may be utilized alone or as part of a pharmaceutical composition as described herein. This, in yet another aspect, the present invention relates to modulator compounds identified by the present methods, and their pharmaceutical compositions.

In the aspects described above, the methods are preferably adapted for implementation in high throughput formats. As used herein, the term “high throughput” refers to the ability to perform at least about at least 100, more preferably about at least 1,000, still more preferably about at least 5,000, and most preferably about at least 10,000, individual assays in a 24 hour period. The term “about” in this context refers to ±10% of a given number.

Likewise, in the aspects described above, the methods are preferably adapted to be carried out in a minimal reaction volume. Preferably, a reaction volume is less than or equal to about 250 uL, more preferably less than or equal to about 100 uL, and still more preferably less than or equal to about 50 uL. The term “about” in this context refers to ±10% of a given number.

As noted above, the assay methods described herein may also be adapted for use as secondary screens, for example, in confirming activity of one or more test molecules identified in an initial screen as binding to isolated PIC components; in lead optimization; in comparing effects of known modulators (e.g., across different viral isolates), etc.

In yet another aspect, the present invention relates to kits for performing the assay methods described above. Such kits preferably comprise at least one target DNA and at least one retroviral preintegration complex, each in an amount sufficient to perform at least one measurement of the integration of retroviral cDNA into a target DNA.

The summary of the invention described above is non-limiting and other features and advantages of the invention will be apparent from the following detailed description of the invention, and from the claims.

### BRIEF DESCRIPTION OF THE FIGURES

FIG. 1 provides a schematic description of a preferred assay format.

FIG. 2 shows various kinetic parameters of the preferred assay format.

### DETAILED DESCRIPTION OF THE INVENTION

The present invention provides novel systems for monitoring retroviral provirus integration. Provirus integration is an attractive target for antiretroviral drugs since integration of viral cDNA is essential for retroviral replication in vivo. Moreover, there are no known cellular enzymes that resemble integrase in sequence or function, so inhibitors of integrase have the potential to otherwise be relatively nontoxic to a host infected with a retrovirus. Additional components in the PIC are also likely to provide additional therapeutic targets, as integrase alone is insufficient to confer complete integration competence to unintegrated viral cDNA. PICs contain multiple proteins in addition to integrase, including the viral MA and RT. NC and Vpr have been detected in some experiments, and the cellular HMG I(Y) proteins and Ku proteins are also present in active PICs. Importantly, PICs display a distinctive response to small molecule inhibitors of integration activity, emphasizing the importance of PIC- based assays in efforts to identify integration inhibitors.

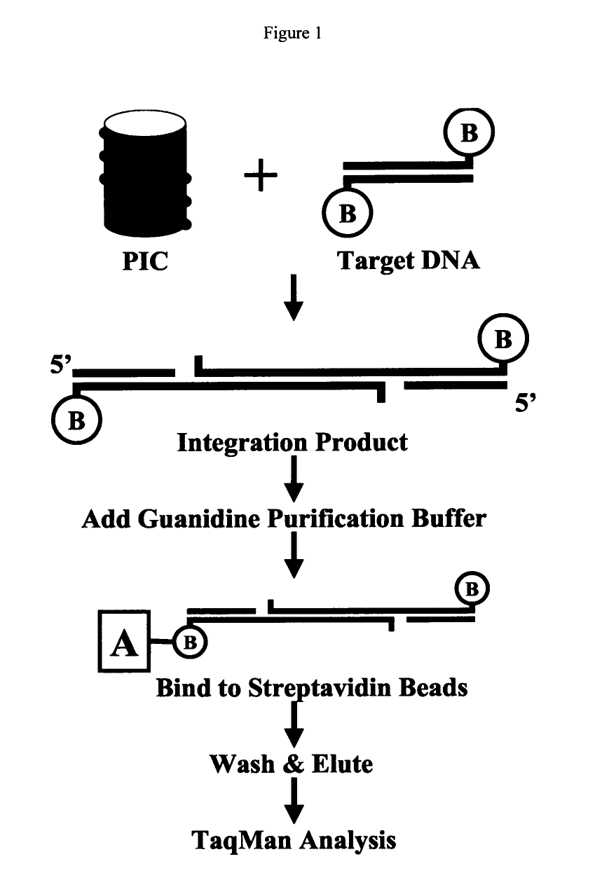

An exemplary form of the assays described herein is illustrated in FIG. 1. As shown in the figure, a linear form of the viral cDNA serves as the immediate precursor of the integrated provirus. Integration of retroviral cDNA requires LTR DNA sites at each end of the unintegrated viral cDNA. Without wishing to be bound to any particular theory, it is believed that, prior to integration, viral integrase removes two nucleotides from the 3′ end of each of the 5′ and 3′ LTRs in the double-stranded viral cDNA, exposing recessed 3′ hydroxyl groups. Integrase is then believed to catalyze attack by the recessed 3′ hydroxyl groups on phosphodiester bonds on each target DNA strand, resulting in joining of each viral DNA 3′ end to protruding 5′ ends in the target. The points of joining on each strand of the target DNA are separated by 5 base pairs. Unfolding of this integration intermediate yields gaps at each junction between viral and host (or target) DNA, and a 5′ two-base flap derived from the viral DNA. Gap repair and ligation of juxtaposed DNA strands are probably carried out by a suitable DNA repair system endogenous to the infected host cell.

Production of Target DNAs

Target DNA can be generated from any suitable source, including plasmid DNA, sheared chromosomal DNA, and from synthetic processes. The target DNA can be any size, although smaller molecules are preferred. Thus, target DNAs can range from as few as about 10 to 5,000 or more nucleotide base pairs in length. For high throughput, small volume reactions, preferred targets range from about 10 to about 200 base pairs in length. Also, as integration appears to be sequence independent, the target can comprise any sequence. For these reasons, particularly preferred target DNAs are generated by annealing synthetic oligonucleotides that have complementary nucleotide sequences. Many techniques for synthesizing or otherwise generating DNA molecules are known in the art.

In the preferred miniaturized PIC assays of the present invention, appropriate short, functional target DNAs are preferably used, as detection by real time PCR (a preferred integration detection technique) may lose efficiency as amplicon length increases, and as increasing target DNA concentrations may inhibit the PCR polymerase used. A comparison of five double-stranded oligonucleotide targets, described hereinafter in the Examples confirmed the sequence-independent nature of retroviral integration, as well as that small targets could be employed. In particular, one target DNA comprised a 60 base pair duplex of mixed nucleotide sequence. Three others comprised 35 base pair duplexes composed of mixed nucleotide sequence, all A:T base pairs, or all G:C base pairs, respectively. Another target DNA comprised a mixed nucleotide sequence 20 base pairs in length. The amount of integration product formed compared in time course assays revealed that functional target DNAs are preferably be between about 15 and about 200 contiguous nucleotides in length. Particularly preferred lengths are in the range from about 60 contiguous nucleotides to about 20 contiguous nucleotides. These target DNAs may comprise either a non-repeating sequence or two or more repeats of the same or different nucleotide sequences. The functional target DNAs used in the invention may also have 5′ or 3′ overhangs (ie., an unpaired base) of one ore more nucleotides at either or both ends of the target DNA molecules (in the case of linear molecules), although double-stranded oligonulceotide-based target DNAs having neither a 5′ nor a 3′ overhang are preferred (such molecules are said to have “blunt” ends).

Despite the preference for the use of small target DNAs having from about 15 to about 200 base pairs, the invention also envisions the use of target DNAs of any size, including those having more than several thousand (e.g., about 2,000, 3,000, 4,000, and 5,000) nucleotide base pairs. Regardless of the length of the target DNA, it is preferred that it contain a single site for integration, and a minimum number (e.g., fewer than about 10, preferably one) of primer binding sites for one of the amplification primer species used in embodiments where integration is detected by a nucleic acid amplification technique. Similarly, while the currently preferred target DNA molecules are linear (ie., neither the 5′ nor 3′ terminal residue located at one end of the molecule is linked to the 5′ nor 3′ terminal residue of the other end of same molecule), the invention contemplates the use of target DNA molecules that have been circularized (i.e., one or both of the 5′ and 3′ terminal residues of one end of the molecule are linked to the 5′ nor 3′ terminal residue of the other end of same molecule, or a repeating unit of the same molecule to produce a concatamer), including those that are supercoiled and those that are not.

As noted above, a tag moiety may be attached to the target DNA molecule and used to isolate or purify the integration products for detection (e.g., by a nucleic acid amplification-based technique such as real-time PCR, by expression of a heterologous marker encoded by the integrated provirus cDNA, etc.). In preferred embodiments, a tag moiety is a member of a binding pair that may be used to purify the tagged DNA. Such a tag moiety is preferably used to purify the target DNA by binding to a complementary binding pair member prior to generating a signal indicative of integration of viral cDNA. A particularly convenient method for purification is to attach to a solid phase a member of a binding pair that is complementary to the other binding pair member (i.e., the tag moiety) on the DNA. The terms “solid phase” and “substrate” refer to a wide variety of materials including solids, semi-solids, gels, films, membranes, meshes, felts, composites, particles, and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Diamandis and T. K. Christopoulos eds., Academic Press: New York, 1996, hereby incorporated by reference. Examples of suitable solid phase substrates include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon, et al., Bioorg. Med. Chem. Lett. 8: 2997 (1998); Kessler, et al., Agnew. Chem. Int. Ed. 40: 165 (2001); Smith, et al., J. Comb. Med. 1: 326 (1999); Orain, et al., Tetrahedron Lett. 42: 515 (2001); Papanikos, et al., J. Am. Chem. Soc. 123: 2176 (2001); Gottschling, et al., Bioorg. And Medicinal Chem. Lett. 11: 2997 (2001).

While a variety of tag moieties are known in the art, preferred tag moieties are members of a binding pair (e.g., antibody/antigen, ligand/receptor, streptavidin/biotin, avidin/biotin, etc.) that may be used to efficiently affinity purify the tagged DNA. Such a tag moiety is preferably used to isolate or purify the target DNA by binding to a complementary binding pair member prior to generating a signal indicative of integration of provirus cDNA. The use of streptavidin magnetic beads is described hereinafter in the exemplary embodiments; however, a wide variety of solid phases and methods for attaching members of binding pairs are known in the art and are suitable for use in the present methods. For instance, other examples of suitable tag moieties include, but are not limited to, biotin, digoxigenin, maltose, oligohistidine, 2,4-dintrobenzene, phenylarsenate, ssDNA, dsDNA, a polypeptide, a metal chelate, and/or a saccharide. Examples of tag moieties and their capture reagents also include but are not limited to: dethiobiotin or structurally modified biotin-based reagents, including deiminobiotin (collectively referred to herein as “biotins”), which bind to proteins of the avidin/streptavidin family (collectively referred to herein as “avidins”); any vicinal diols, such as 1,2-dihydroxyethane, and other 1,2-dihyroxyalkanes including those of cyclic alkanes, e.g., 1,2-dihydroxycyclohexane which bind to an alkyl or aryl boronic acid or boronic acid esters, such as phenyl-B(OH)₂ or hexyl-B(Oethyl)₂; maltose which binds to maltose binding protein (as well as any other sugar/sugar binding protein pair; a hapten, such as the dinitrophenyl group, to which an antibody can be generated; a molecule which binds to a transition metal, for example, an oligomeric histidine will bind to Ni(II), the transition metal capture reagent may be used in the form of a resin bound chelated transition metal, such as nitrilotriacetic acid-chelated Ni(II) or iminodiacetic acid-chelated Ni(II); glutathione which binds to glutathione-S-transferase.

Production of PICs

Preintegration complexes (PICs)—integration-competent viral replication intermediates—can be partially purified from freshly infected cells and used as a source of integration activity in vitro. See, e.g., Brown, et al., Cell 49: 347-56, 1987; Ellison, et al., J. Virol. 64: 2711-15; Farnet and Haseltine, Proc. Natl. Acad. Sci. USA 87:4164-68, 1990; and Lee and Coffin, J. Virol. 64: 5958-5965, 1990. PICs contain multiple proteins in addition to integrase. For example, the retroviral proteins MA, RT, NC, and Vpr have been detected in some experiments, and the cellular HMG I(Y) proteins and Ku proteins are also typically present. The present invention may also be adapted to identification of additional components of the PIC that may represent additional therapeutic targets. For example, salt washing may be used to selectively remove components of the PIC, and characterized or uncharacterized fractions (either from washed PICs or simply factors obtained from cells such as T-cells) added back in experiments to assess reconstitution of full PIC function. See, e.g., Brooun, et al., J. Biol. Chem. 276: 46946-52, 2001. Those components necessary for reconstitution may then be further subjected to the modulator identification methods described hereinafter.

Detection of Provirus Integration

Provirus integration may be observed in the integration products by methods well known in the art. Detection may be by simple hybridization (e.g., Southern blotting or filter blotting; see, e.g., Farnet, et al., Proc. Nat'l. Acad Sci USA 93: 9742-47, 1996) in low throughput methods, by a variety of amplification/hybridization assays known in the art, as well as by detecting signals produced by markers engineered for inclusion in viral cDNA.

In preferred embodiments, integration of viral cDNA is detected by a nucleic acid amplification technique adapted for the particular assay. In general, after incubation, target DNAs are separated from the other reaction components, particularly unintegrated viral cDNA molecules. Integration events can then be detected by amplifying a region of the integrated provirus alone, or by detecting one or more of the integration junctions. As the nucleotide sequences of both the target DNA and engineered viral cDNA are known, suitable amplification primers can be readily designed.

For increased throughput, a “real time PCR” assay providing dynamic, and preferably quantitative, fluorescence detection of amplified products may be used. During real time PCR, the amplified products hybridize to probe nucleic acids, which are labeled with both a reporter dye and a quencher dye. When these two dyes are in close proximity, i.e., both are present in an intact probe oligonucleotide, the fluorescence of the reporter dye is suppressed. However, a polymerase, such as AmpliTaq Gold™, having 5′-3′ nuclease activity, can be provided in the PCR reaction. This enzyme cleaves the fluorogenic probe if it is bound specifically to its intended nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye can be detected and/or quantified. The increase in fluorescence is a direct consequence of amplification of target nucleic acids during PCR. Real time PCR is distinguished from “end point” PCR, where a signal is generated only at the end of the amplification procedure rather than during the amplification cycles.

It is preferred to minimize the number of manipulations involved in the present assays. Thus, while target DNAs containing integrated viral cDNA are preferably purified (e.g., by affinity chromatography) prior to the detection step, additional purification steps (e.g., treatment with proteinase K or an exonuclease (such as λ exonuclease in embodiments where the target DNAs are linear and have 5′ overhangs), phenol extraction, and/or ethanol precipitation) prior to detection (e.g., by real time PCR) are preferably not used, as such methods require rather extensive manipulation and are not preferred for a high throughput screen. That said, it has been discovered that a particularly efficient clean up step using a chaotropic agent can be used, alone or in combination with a filtration step, prior to the detection step. For example, Millipore™ filter purification columns may be used, in which samples are brought to a guanidine hydrochloride concentration of 2 M, bound to a Millipore™ filter purification column, washed, and eluted. Although somewhat time consuming, such a step is adaptable to a multiplex format, such as in a microtiter plate, and so is suitable for inclusion in high throughput assays.

In such methods, one key parameter for good product recovery may not be purification on the column but rather the treatment with a chaotropic agent (e.g., guanidine hydrochloride, urea, etc.). Indeed, it has been discovered that a simple treatment of the integration reaction with a chaotropic agent such as guanidine hydrochloride substantially increases yield, particularly when followed by tag-based purification (e.g., with an affinity solid phase such as the magnetic beads described in the examples), which results in excellent product recovery and a sufficiently high signal to noise ratio.

As noted above, some purification processes may involve treatment of integration reactions with an exonuclease, for example, λ exonuclease, which degrades linear double-stranded DNAs that have 5′ overhangs. While the activity of such nucleases may be inhibited by proteins bound to the DNA, such processes are not preferred in assays where the detection step involves a nucleic acid amplification step or expression of a marker gene encoded by the engineered provirus. Thus, methods of the invention that do not involve degradation of nucleic acid molecules in the reaction are said to require no pre-treatment to degrade nucleic acids.

Tag-based affinity purification can be accomplished by any suitable method. For example, one or more tag moieties can be attached at the ends of target DNA molecules (5′ and/or 3′) as well as on non-terminal nucleotides. The location of the tag moiety on the target DNA oligonucleotide may have an effect on the efficiency of product DNA recovery. Integration of unintegrated viral cDNA by PICs in vitro results in formation of an integration intermediate in which only one DNA strand is connected at each host-virus DNA junction. The polarity of joining to the tagged oligonucleotide is such that the target DNA 3′ ends become covalently bound to the viral cDNA, while the target 5′ end is attached by DNA strand annealing only. 3′-modified target DNAs may be much more efficient targets, as the difference in product recovery seen for the different positions of modification may be because the short strands of target DNA in the 5′-modified substrate do not stay stably annealed during binding to the beads and washing. Denaturation of this strand would separate the provirus cDNA from the modified target DNA, resulting in loss of the integration product. Consequently, 3′-modified target DNAs are preferred.

Screening Methods for PIC-Dependent Integration Modulators

Screening methods for modulators of PIC-dependent integration are exemplified herein by testing sensitivity to known integrase inhibitors. The diketo acid family of inhibitors has been shown to inhibit HIV replication by targeting integrase in vivo. See, e.g., Hazuda, et al., Science 287: 646-50, 2000. Members of this family have also been shown to inhibit PICs in vitro. To assess potential modulators using the assays described herein, reactions may be exposed to various concentrations of such diketo acid inhibitors (e.g., LXXX616, Hazuda, et al., supra). Such potential modulators may be contacted with PICs before or simultaneously with contacting the PICs with the target DNA. Likewise, in those methods where components are added to inactive PICs to reconstitute active PIC function, those factors may be contacted with potential modulators prior to reconstitution, simultaneously with reconstitution, or following reconstitution.

Selection of Lead Molecules

Certain potential classes of lead compounds for inhibition of provirus integration have been identified (e.g., anthraquinones in Famet, et al., Proc. Natl. Acad. Sci. USA 93: 9742-47, 1996; these may serve as initial chemical backbones for design of new integration modulators). In addition, a variety of techniques are available in the art for generating combinatorial libraries of small organic molecules. See generally Blondelle, et al., Trends Anal. Chem. 14: 83, 1995; U.S. Pat. Nos. 5,359,115, 5,362,899, 5,288,514, and 5,721,099; Chen, et al., JACS 116: 2661, 1994; Kerr, et al., JACS 115: 252, 1993; WO92/10092, WO93/09668, WO 94/08051, WO93/20242, and WO91/07087. A variety of libraries on the order of about 16 to 1,000,000 or more chemically diverse molecules can be synthesized and screened for a particular activity or property using such methods. As described hereinafter, new integrase inhibitors were characterized by the methods of the present invention, derived from a combinatorial library of compounds derived from modified triazines. The most potent of these, ACV 351 and ACV 343, inhibited active HIV PICs with IC50s of 9 uM and 4.7 uM, respectively.

Antibody or antibody fragments may also find use in the methods described herein. In such embodiments, antibodies may be generated to various components of the PIC, which antibodies can then be screened to identify those that modulate PIC-mediated integration. The term “antibody” as used herein refers to a peptide or polypeptide derived from, modeled after, or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994,) J. Immunol. Methods 175:267-273; Yarmush (1992). J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward, et al., (1989) Nature 341:544-546), which consists of a VH domain; and (vi) an isolated complementarity determining region (CDR). Single chain antibodies, monoclonal antibodies, polyclonal antibodies, and antibodies obtained by molecular biological techniques (e.g., by phage display methods) are also included by reference in the term “antibody.” Preferred antibodies are “Omniclonal” antibodies, which means a mixture of different antibody molecules selected from a phage display library, where each antibody specifically binds to a target antigen with a minimum affinity of 10⁹ M⁻¹ to 10¹⁰ M⁻¹

The generation and selection of antibodies may be accomplished several ways. For example, selected polypeptides may be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g., Cwirla, et al., Proc. Natl. Acad. Sci. USA 87, 6378-82, 1990; Devlin, et al., Science 249, 404-6, 1990, Scott and Smith, Science, 249, 386-88, 1990; and Ladner, et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material allows simultaneous mass screening of very large numbers of phage bearing different polypeptides. Phage displaying a polypeptide with affinity for a target can bind to the target, and these phage can then be enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098.

Antibodies that are generated by these methods may then be selected by first screening for affinity and specificity with the purified target protein of interest. The screening procedure can involve immobilization of the purified target protein in separate wells of microtiter plates. The solution containing a potential antibody or groups of antibodies is then placed into the respective microtiter wells and incubated for about 30 min. to 2 h. If an antibody to the protein(s) of interest is present in the solution, it will bind to the immobilized protein(s). The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized natriuretic fragment(s) is present. The antibodies so identified may then be further analyzed for the ability to modulate PIC-mediated provirus integration in the assays described herein.

Preparation of Pharmaceutical Compositions

Modulators of the present invention may be administered as antiretroviral agents to subjects, including humans and non-human animals. When administered, the pharmaceutical preparations of the invention are applied in pharmaceutically acceptable amounts and in pharmaceutically acceptable compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts. The pharmaceutical compositions also may contain, optionally, suitable preservatives, such as: benzalkonium chloride; chlorobutanol; parabens; and thimerosal. Carrier formulations suitable for oral, subcutaneous, intravenous, intramuscular, etc. administrations can be found in Remington's Pharmaceutical Sciences, Mack Publishing Co., Easton, Pa.

A variety of administration routes are available for treating a subject. The particular mode of delivery selected will depend upon the particular compound selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, intravenous or parenteral routes. Such modes of administration also include obtaining T cells or bone marrow cells, stem cells, or early lineage progenitor cells from a patient and contacting the isolated cells with the compounds of the invention ex vivo, followed by reintroducing the treated cells to the patient. The treated cells can be reintroduced to the patient in any manner known in the art for administering viable cells.

Oral administration is particularly preferred. Compositions suitable for oral administration may be presented as discrete units, such as capsules, tablets, lozenges, each containing a predetermined amount of the compound of the invention. Other compositions include suspensions in aqueous liquids or non-aqueous liquids such as a syrup, elixir, or an emulsion. Preferably, the oral preparation does not include an enteric coating since it is desirable to expose the cyclic compounds of the invention to the acidic pH conditions of the digestive tract to convert the cyclic molecules to their linear counterparts.

Other delivery systems can include time-release, delayed release, or sustained release delivery systems. Such systems can avoid repeated administrations of the compounds described above, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids, including sterols such as cholesterol, cholesterol esters, and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the compound is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, as used herein, means that the implant is constructed and arranged to delivery therapeutic levels of the active ingredient for at least 10 days, and preferably 60 days. Long-term sustained release implants are well known to those of ordinary skill in the art and include some of the release systems described above.

The selected compounds are administered in effective amounts. An effective amount is a dosage of the compound sufficient to provide a medically desirable result. The effective amount will vary with the particular condition being treated, the age and physical condition of the subject being treated, the severity of the condition, the duration of the treatment, the nature of the concurrent therapy (if any), the specific route of administration and like factors within the knowledge and expertise of the health practitioner. Generally, doses of active compounds will be from about 0.001 mg/kg per day to 1000 mg/kg per day. It is expected that doses range of 0.001 to 100 mg/kg will be suitable, preferably orally and in one or several administrations per day. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

### EXAMPLES

The following examples serve to illustrate the present invention. These examples are in no way intended to limit the scope of the invention.

### Example 1

### HIV PIC Extract Preparation

To prepare HIV PICs, HIV-based vector stocks were prepared from the SKSM2 cell line as described by Hansen, et al. (Nat. Biotechnol., 17: 578-82, 1999). Briefly, the SKSM2 cell line encodes 1) HIV gag and pol synthesized from an RNA that cannot be packaged in particles, 2) the VSV-G envelope protein, and 3) a packagable, replication defective vector component transducing gfp (green fluorescent protein) and containing all the sites need in cis for reverse transcription and integration. Induction of viral particle production was started on day 0 by removal of doxycycline, sodium butyrate was added on day 1, and virus was harvested on days 2-6. Virus concentration was determined by measuring p24 antigen.

Cells for HIV production (293T) were grown to 80% confluence on 15cm² plates and were infected at an m.o.i of roughly 10 in the presence of 10 ug/ml DEAE dextran (Sigma, St. Louis, Mo.). Six hours after infection, cells were washed with buffer K (150 mM KCl, 20 mM HEPES, pH 7.4, 5 mM MgCl₂), then permeabilized in 0.5 mL of buffer K per plate with 0.05% NP40 (Calbiochem, La Jolla, Calif.) for 10 minutes on ice. Lysates were centrifuged at 2200 rpm, and PIC-containing supernatant was centrifuged at 10,000 rpm and then stored at 80° C.

### Example 2

### Preparation of Integration Target DNA

Exemplary target DNAs used in these methods are provided in Table 1, below:

| | |
|---|---|
| Oligonucleotides | Sequence |

| | |
|---|---|
| Target DNA, 3′bio60, Forward | 5′GCCGAATTCGAATTCCGAATATGCAAGC |
| | GTTACGTAACGTTACGCAAGCTTAAGCTTG |
| | CG-3′biotin |
| Target DNA, 3′bio60 Reverse | 5′CGCAAGCTTAAGCTTGCGTAACGTTACGT |
| | AACGCTTGCATATTCGGAATTCGAATTCGG |
| | C-3′biotin |
| Target DNA, 3′bio35, Forward | 5′GCCCGAATATGCAAGCGTTACGTAACGT |
| | TACGCGC-3′biotin |
| Target DNA, 3′bio35, Reverse | 5′GCGCGTAACGTTACGTAACGCTTGCATAT |
| | TCGGGC-3′biotin |
| Target DNA, 3′bioAT, Forward | 5′AAT TAT ATT TAT AAA TAT TTA TAT |
| | TAA TTT TAT AT-3′biotin |
| Target DNA, 3′bioAT, Reverse | 5′ATA TAA AAT TAA TAT AAA TAT TTA |
| | TAA ATA TAA TT-3′biotin |
| Target DNA, 3′bioGC, Forward | 5′CCG GGC GCC CGG CCC GGG CGC GCC |
| | GGG CGC GC-3′biotin |
| Target DNA, 3′bioGC, Reverse | 5′GCG CGC CCG GCG CGC CCG GGC CGG |
| | CCG GCG CCC GG-3′biotin |

### Example 3

### PIC Integration Reaction

Magnetic beads comprising immobilized streptavidin (Dynal, Oslo, Norway) were rinsed three times with PBS, blocked for 1 hour with Blocking Buffer (200mM NaCl, 20 mM HEPES, pH7.4, 5 mM EDTA, 0.2% Triton X-100, 0.4 mg/ml Sonicated Salmon Sperm DNA (Stratagene, Cedar Creek, Tex.), 0.2% BSA). Blocked beads were rinsed once with PBS and then once with Dynal B&W Buffer (2 M NaCl, 10 mM Tris-HCl, pH 7.5, 1 mM EDTA). The beads were then resuspended in 50 uL Dynal B&W Buffer per integration reaction, and added to a 96-well vinyl plate (Corning, Cambridge, Mass.). 50ug of streptavidin magnetic beads were used per integration reaction.

Integration reactions were carried out in 96-well polypropylene plates, with 2 uL PIC extract and 50ng of 3′-biotinylated oligonucleotide as target DNA. The addition of buffer K, 10% glycerol, and 0.1% Triton X-100 was used to bring the reaction up to a final volume of 100 uL. Reactions were incubated for 30 minutes at 37° C. to allow for integration. To break apart the preintegration complexes, 50 uL of purification buffer (5.4 M Guanidine HCl, 150 mM KOAc, pH 4.8) was added to the integration reactions, and incubated at room temperature for 5 minutes.

The integration reactions with purification buffer were then transferred to a vinyl plate containing streptavidin magnetic beads, and incubated for 1 hour with shaking at room temperature to allow for binding. The binding solution was then removed, and the beads were washed three times with 100 uL Wash buffer (1 M NaCl, 0.1% Triton X-100), with 5 minute shaking incubations between washes, and rinsed twice with 50 ul rinse buffer (100 mM NaCl, 20 mM HEPES, pH 7.4). Samples were eluted by the addition of 25 ul 0.04 N NaOH and 0.1% Tween-20 at 50° C. for 10 minutes. Eluted product was then transferred to a new microtiter plate and neutralized by the addition of 25 ul 40 mM HCl and 50 mM HEPES, pH 7.4.

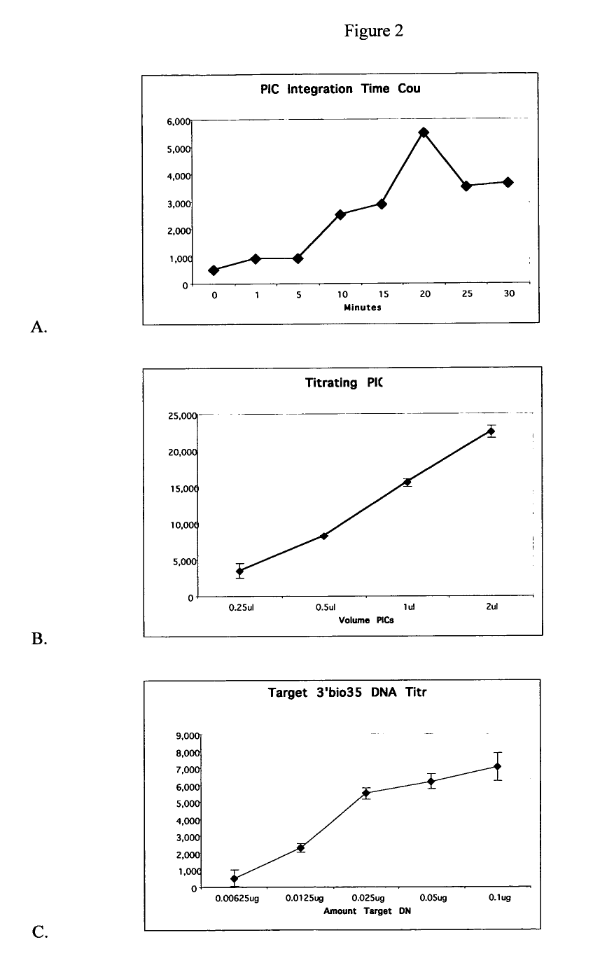

Some characteristics of the miniaturized PIC assay are shown in FIG. 2. Time course analysis indicates that the reaction was about half complete after 10 min, and completed after about 20 min. (FIG. 2A). The amount of reaction product generated was linear with input amount of PICs over a range of 2 uL to 0.25 uL input PIC extract (FIG. 2B). Even with as little as 0.25 uL PIC extract the signal was still 5-fold above the background. This is in contrast to previously reported assays, in which much larger volumes of PIC extracts were required. A titration of target DNA showed that 0.1 ug per reaction of the biotinylated target DNA was preferred. (FIG. 2C).

### Example 4

### Screening of PIC Integration Inhibitors

Compounds were tested for the ability to inhibit integration of PIC viral DNA essentially as described in Example 3. When testing inhibitors, 5% DMSO was added to a positive control reaction without inhibitor, since the test compounds were dissolved in DMSO. In inhibitor studies, 5 uL of 20× drug stocks in DMSO were incubated with the PIC extract for 10 minutes at room temperature before addition of the target DNA.

### Example 5

### Detection of Integration Using Quantitative PCR

Fluorescence-monitored real-time PCR (TaqMan) reactions were performed on an ABI Prism 7700 (PE Applied Biosystems) in a volume of 25 uL. Primers for amplification recognized the Late RT sequence of the SKSM2 HIV vector, and are found in Table 2, below (IDT, Coralville, Iowa). Reactions contained 300 nM primers, 100 nM probe, 1× master mix (PE Applied Biosystems), and 5 uL of integration product. The cycling method was 95° C. for 10 min, and 40 cycles of 95° C. 15 s, and 60° C. for 1 min. Primers used in these assays are provided in Table 2, below:

| | |
|---|---|
| Late RT Forward Primer: | 5′-TGT GTG CCC GTC |
| TaqMan | TGT TGT GT-3′ |
| Late RT Reverse Primer: | 5′-GAG TCC TGC GTC |
| TaqMan | GAG AGA GC-3′ |
| Late RT Probe: | 5′-/56-FAM/CAG TGG |
| TaqMan | CGC CCG AAC AGG |
| | GA/36-TAMNph/-3′ |

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A method of detecting integration of an engineered viral cDNA into a free-floating target DNA, comprising:
(i) in an aqueous reaction, contacting said free-floating target DNA with a retroviral preintegration complex comprising said engineered viral cDNA under conditions that allow integration of viral cDNA from an active retroviral preintegration complex into said target DNA; and
(ii) detecting whether said engineered viral cDNA integrated into said target DNA.

2. A method according to claim 1, wherein said engineered viral cDNA includes at least one of the features selected from the group consisting of lacking replicative capacity and encoding a heterologous marker.

3. A method according to claim 1, wherein detection of integration is accomplished by identifying a nucleic acid molecule indicative of integration of the engineered viral cDNA into said target DNA.

4. A method according to claim 3, wherein the identification step is accomplished by amplifying a nucleic acid molecule indicative of integration of the engineered viral cDNA into said target DNA.

5. A method according to claim 1, wherein said retroviral preintegration complex is selected from the group consisting of a lentivirus preintegration complex, a human lentivirus preintegration complex, an HIV preintegration complex, and an oncoviral preintegration complex.

6. A method according to claim 1, wherein said retroviral preintegration complex is obtained from cells infected by a replication defective retrovirus.

7. A method according to claim 1, wherein said target DNA comprises a tag moiety.

8. A method according to claim 7, wherein said tag moiety comprises a first member of a binding pair, and wherein said method further comprises purifying said target DNA by binding said first member to its complementary binding pair member prior to said detecting step (ii).

9. A method according to claim 8, wherein said first member of the binding pair is selected from the group consisting of a hapten, an antibody, a receptor, a receptor ligand, avidin, streptavidin, and biotin.

10. A method according to claim 8, wherein said first member is streptavidin or biotin.

11. A method according to claim 8, wherein said complementary binding pair member is immobilized to a solid phase.

12. A method according to claim 1, wherein said target DNA is a DNA duplex comprised of two complementary oligonucleotides.

13. A method according to claim 12, wherein said DNA duplex has a length selected from the group consisting of between about 15 to about 2,000 base pairs, between about 15 to about 200 base pairs, and about 20 base pairs.

14. A method according to claim 1 comprising treating the reaction with a chaotropic agent prior to detecting whether said engineered viral cDNA integrated into said target DNA.

15. A method according to claim 1, wherein the retroviral preintegration complex of step (i) is an active retroviral preintegration complex, and wherein said method further comprises contacting said active retroviral preintegration complex with one or more test molecules to be screened for integration modulating activity prior to or concurrently with said contacting step (i), and comparing the signal from said signal generating step (ii) to a baseline signal obtained by performing said method in the absence of said one or more molecules.

16. A method according to claim 15 that is a high throughput mehtod.

17. A method according to claim 1 carried out in a reaction volume of less than about 100 μL.

18. A kit for performing the method of claim 1, comprising at least one target DNA and at least one retroviral preintegration complex, each in an amount sufficient to perform at least one measurement of the integration of retroviral viral cDNA into a target DNA.

19. A method of identifying a compound that modulates integration of a provirus, comprising:
(i) in solution, contacting a test compound with an active retroviral preintegration complex that comprises an engineered viral cDNA;
(ii) concurrent with or after contacting the test compound with the active retroviral preintegration complex, adding a free-floating target DNA to the solution of part (i) under conditions that allow integration of a viral cDNA from an active retroviral preintegration complex into the target DNA; and
(iii) determining whether said viral cDNA integration into the target DNA is modulated by the test compound and, if so, identifying the test compound as a modulator of integration of a provirus.

20. A method of detecting integration of an engineered viral cDNA into a free-floating target DNA, comprising:
(i) in an aqueous reaction, contacting said free-floating target DNA with a retroviral preintegration complex comprising said engineered viral cDNA under conditions that allow integration of viral cDNA from an active retroviral preintegration complex into said target DNA; and
(ii) detecting whether said engineered viral cDNA integrated into said target DNA, wherein the detecting does not require pre-treatment with an exonuclease.
